(19) United States
(12) Patent Application Publication
(10) Pub. No.: US 2014/0314848 A1
(43) Pub. Date: Oct. 23, 2014

(54) PHARMACEUTICAL COMPOSITIONS OF ANTIHYPERTENSIVES

(71) Applicant: EMS S.A., Hortolandia (BR)

(72) Inventors: Giuseppe Soldati, Hortolandia - SP (BR); Giancarlo Santus, Hortolandia - SP (BR); Ricardo Vian Marques, Hortolandia - SP (BR); Leticia Khater Covesi, Hortolandia - SP (BR); Pedro Bordeaux Rego, Hortolandia - SP (BR)

(21) Appl. No.: 14/348,643

(22) PCT Filed: Oct. 2, 2012
(86) PCT No.: PCT/EP2012/069401

§ 371 Date: Mar. 31, 2014

(30) Foreign Application Priority Data
Oct. 3, 2011 (GB) 1116993.5

(51) Int. Cl. A61K 31/4178; A61K 9/20; A61K 31/404

(52) U.S. Cl. CPC A61K 31/4178; A61K 31/404; A61K 9/2086; A61K 9/2095
USPC 424/472; 514/381; 264/113

(57) ABSTRACT

The present invention describes antihypertensive pharmaceutical formulations particularly formulations of losartan and indapamide.

### FIELD OF THE INVENTION

This invention relates to the controlled release of pharmaceutical compositions comprising formulations of antihypertensive drugs in particular to formulations comprising indapamide and losartan with controlled release of the active ingredients to provide efficient control of blood pressure and reduced side effects.

### BACKGROUND

Hypertension is the term used to describe high blood pressure that is a measurement of he force against the walls of arteries as the heart pumps blood through the body.

Blood pressure readings are measured in millimeters of mercury (mmHg) and usually given as two numbers—for example, 120 over 80 (written as 120/180 mmHg). The top number is the systolic pressure that is considered high if it is over 140 mmHg most of the time. The bottom number is the diastolic pressure that is considered high if it is over 90 mmHg most of the time.

The goal of hypertensive treatment is to reduce blood pressure in order to lower risk of complications. There are many different drugs that can be used to treat high blood pressure, including: alpha blockers, angiotensin-converting enzyme (ACE) inhibitors, angiotensin receptor blockers (ARBs), beta blockers, calcium channel blockers, central alpha agonists, diuretics, renin inhibitors, vasodilators.

Often, a single blood pressure drug may not be enough to control hypertension because achieving its targets can be challenging. From a clinical perspective, fixed-dose antihypertensive combinations offer certain advantages in terms of efficacy, adherence, cost, convenience, patient-perceived ‘wellness’ and side effects. Consequently, in the future, fixed-dose combination formulations are likely to become increasingly used in the treatment of cardiovascular disease (Lewanczuk and Tobe, 2007).

Losartan is an antagonist of ANGIOTENSIN type 1 receptor with antihypertensive activity due to the reduced pressure effect of ANGIOTENSIN II. Losartan was the first of a class of antihypertensive agents called angiotensin II receptor antagonists. It was developed initially for the reduction of the combined risk of cardiovascular death, heart attack and stroke in patients with hypertension and left ventricular hypertrophy and then for the oral treatment of hypertension.

Indapamide is a non-thiazide sulphonamide diuretic drug indicated for the treatment of hypertension, alone or in combination with other antihypertensive drugs as well as for the treatment of salt and fluid retention associated with congestive heart failure or edema from pregnancy. Commonly reported adverse events are hypokalemia (low potassium levels), fatigue and orthostatic hypotension. Many of the adverse events are associated to electrolyte abnormalities and this can be prevented with a controlled and gradual release of indapamide from the dosage form.

On the other hand, losartan can significantly decrease serum uric acid levels by augmenting uric acid excretion and Nike et al, 2000 have confirmed the mild uricosuric and hypouricaemic effect of losartan, whereas for the first time, it is shown that combination therapy with indapamide and losartan is not associated with any change in serum uric acid levels.

There are several references known in the literature, which describe different combinations of ANGIOTENSIN II AT-1 RECEPTOR ANTAGONISTS with other pharmacological class. International Publication WO94/09778 describes the combination of ANGIOTENSIN II RECEPTOR ANTAGONISTS and diuretics. International Publication No. (PCT) WO 03097045 describes the combination of an angiotensin receptor blocker with a calcium channel blocker and a diuretic. International Publication WO89/06233 reports combinations of ANGIOTENSIN II receptor blockers with diuretics and NSAIDs.

international Publication WO2003/059327 discloses bilayer tablets comprising telmisartan and hydrochlorothiazide displaying fast dissolution and immediate drug release profile. International Publication WO2005/009413 describes tablets that include a prolonged release core and an immediate release layer with several categories of drugs.

Although combinations of drugs specifically in the hypertension field are known, there is still so a lack of a specific drug combination providing an advantage during patient treatment with good bioavailability, reduced side effects and easy to produce with industrial machines. Despite the various types of combination products dosage forms conceivable it cannot be predicted which of these dosage forms best combines product stability, pharmacological efficacy, and a reliable manufacturing method.

It is an object of the present invention to provide a novel tablet dosage form, which can encompass drugs of different classes which otherwise pose stability issues in a single unit.

Another approach of the present invention is to provide a combination therapy of losartan with a diuretic such as indapamide to achieve the synergistic therapeutic efficacy required in the treatment of hypertension.

The present invention therefore provides the combination of an ANGIOTENSIN II AT-1 RECEPTOR ANTAGONISTS with a controlled release diuretic to treat cardiovascular and in particular blood pressure related disorders.

### SUMMARY OF THE INVENTION

One embodiment of the present invention is a dual layer tablet of an ANGIOTENSIN II AT-1 RECEPTOR ANTAGONISTS inhibitor with a diuretic far achieving an immediate release of the ANGIOTENSIN II AT-1 RECEPTOR ANTAGONISTS inhibitor and a modified release of the diuretic.

A second embodiment of the present invention is the use of the combination of an ANGIOTENSIN II AT-1 RECEPTOR ANTAGONISTS inhibitor with a controlled release diuretic to treat cardiovascular and in particular blood pressure related disorders.

A third embodiment of the present invention is the process to formulate ANGIOTENSIN II AT-1 RECEPTOR ANTAGONISTS inhibitors with controlled release diuretics.

In the preferred embodiments of the invention the ANGIOTENSIN II AT-1 RECEPTOR ANTAGONISTS inhibitor is losartan potassium and the diuretic is indapamide.

The dosage form is preferably a tablet which may include a coating. In the preferred embodiments the tablet may be coated with one or more polymers or pharmaceutically acceptable seal coat polymers. The tablet may form a bilayered tablet. The pharmaceutically acceptable excipients within the tablet may include one or more of binders, fillers, so antioxidants, disintegrants, surfactants, lubricants and glidants and the like. Alternatively the two materials may be included in a capsule.

### DESCRIPTION OF THE INVENTION

The present invention provides pharmaceutical compositions of an ANGIOTENSIN II AT-1 RECEPTOR ANTAGONISTS inhibitor, such as losartan potassium, and a diuretic, such as indapamide, for the treatment of hypertension, wherein said composition provides improved patient treatment, has good bioavailability and causes reduced systemic side effects.

In, a preferred embodiment the present invention deals with a dual-layer tablet comprising a layer of fast release dose of an ANGIOTENSIN II AT-I RECEPTOR ANTAGONIST such as losartan potassium and a layer with a controlled release dose of a diuretic such as indapamide. The two layers are tableted together in a single tablet.

Upon ingestion and contact with gastrointestinal fluids, the preferred dual-layer tablet releases quickly losartan potassium from one layer and, in a gradual and controlled manner, releases indapamide from the other layer.

The release of the active ingredient can be followed with standard dissolution test. For example, with the dissolution test described by United States Pharmacopeia (USP)—apparatus 2 (paddle), at 50 rpm, in buffer pH 6.8 in 900 ml—more than 75% of losartan potassium is released in 30 minutes from the fast release layer, while the release of indapamide from the controlled release layer was around 20% after 6 hours, around 40% after 12 hours and around 60% after 24 hours.

Alternatively to the dual-layer tablet, the combination of the two active ingredients, one released quickly (losartan) and one in a controlled way (indapamide) may be obtained by filling a capsule with an fast release granulate of losartan and one or more) controlled release tablet(s) or pellets of indapamide.

Components of the losartan fast release layer of the tablet or of the capsule generally comprises diluents (in an amount in the range of 10 wt % to 60 wt %, preferably 15 wt % to 40 wt %) such as lactose, mannitol, starch, microcrystalline cellulose, etc, and lubricants and glidants (in an amount in the range of 0.01 wt % to 5 wt %, preferably 0.1 wt % to 2 wt %) such as magnesium stearate, talc, colloidal silicon dioxide, binders (in an amount in the range of 1 wt % to 20 wt %, preferably 2 wt % to 10 wt %) such as povidone, modified starch, PVA, disintegrants (in an amount in the range of 0.1 wt % to 20 wt %, preferably 1 wt % to 5 wt %) such as so called cross-linked sodium carboxymethylcellulose (croscarmellose sodium) Ac-Di-Sol®, Nymcel etc. Examples of materials that can be employed are reported in Remington “The science and practice of Pharmacy” 21st edition, 2005 and in the “Handbook of Pharmaceutical Excipients” 2006, 5th edition.

Other excipients or additives may be added to the formulation to enhance the efficacy of the active ingredient, to reduce the side effects and/or toxic effects, to prolong the duration of the active ingredient in the systemic circulation. Additional ingredients may also be added to the formulation which enhance the stability of the active pharmaceutical ingredient or formulation, such as anti-oxidants. Still other ingredients may be added to the formulation, such as colourings, flavourings, sweeteners and the like to enhance the receptivity and compliance by patients or other users of the formulations.

The same excipients can be used for the controlled release layer with the inclusion of excipients capable to control the release of the active ingredient. Preferred control release excipients are cellulose derivatives in particular hydroxypropylmethylcellulose (HPMC) preferably in an amount in the range of 10 wt % to 50 wt % preferably 20 wt % to 40 wt %.

The present invention may be used for the improved bioavailability and tolerability of any ANGIOTENSIN II AT-1 RECEPTOR ANTAGONISTS inhibitor and any diuretic. Although it has been described in relation to the preferred ANGIOTENSIN II AT-1 RECEPTOR ANTAGONISTS inhibitor losartan potassium and diuretic indapamide it can be used with any ANGIOTENSIN II AT-1 RECEPTOR ANTAGONISTS inhibitor and any diuretic.

These antihypertensive drugs may be contained within a tablet or within a capsule or a sachet in a solid dosage forms capable to enhance bioavailability and improve tolerability.

Potential advantages of the present invention are to increase patient medication adherence, to reduce side effects and individual and system costs by reducing the number of tablets or capsules and the number of daily dosage forms administrations.

The preferred compositions of the invention are administered at a dose in the range of 10 to 200 mg/day of losartan and 0.5 to 5 mg/day of indapamide. The recommended dose in adult hypertensive patients is 50 mg twice daily (BID) for losartan potassium and 1.5 mg once a day for indapamide controlled release.

A common dosage and administration that can be anticipated is of a once a day dual-layer so tablet of 50 or 100 mg of losartan potassium and 1.5 mg of indapamide.

The pharmaceuticals compositions of the present invention could be administered in combination with other antihypertensive drugs such as: alpha blockers, angiotensin receptor blockers (ARBs), beta blockers, calcium channel blockers, central alpha agonists, renin inhibitors, vasodilators.

The present invention will now be illustrated by the following examples. It is understood, however, that such examples are provided for illustration only, and the invention is not intended to be limited by the examples. The formulation based on the system employed in the examples can be formed by any suitable method known in the art.

### EXAMPLE 1

### Preparation of Losartan Potassium Layer

### Composition of the Layer

| | | |
|---|---|---|
| Losartan potassium micronized | 100.0 | mg |
| Lactose monohydrate | 51.0 | mg |
| Cellulose microcrystalline | 105.0 | mg |
| Pregelatinized starch | 41.9 | mg |
| Magnesium stearate | 2.1 | mg |
| Water (removed during the process) | 17.2 | mg |

### Procedure

Losartan potassium, cellulose microcrystalline, lactose monohydrate and pregelatinized maize starch were sieved and blended for few minutes.

The mixture of powders was kneaded using a solution of purified water and pregelatinized maize starch, and the wet granulate was passed through a 2.0 mm sieve.

The granulate was dried in a static oven at 50° C., until a LOD≦4% is reached, then passed through a 0,6 mm sieve.

Remaining excipient (magnesium stearate) was sieved and added.

After blending, final mixture was compressed to the target weight (300 mg for 100 mg strength and 150 mg for 50 mg strength) as the second layer of the double layer tablet, to the hardness range of 15-20 kp.

### Technological Controls

Technological controls of the granulate and of the tablets are reported in the table below:

Technological characterization: granulate

| | | |
|---|---|---|
| Fowability (flow through an orifice) | 5.6 | g/sec (orifice 10.0 mm) |
| | 15.40 | g/sec (orifice 15.0 mm) |
| Compressibility Index | 11.11 | (good) |
| Hausner Ratio | 1.13 | (good) |

Technological characterization: tablet

| | |
|---|---|
| Hardness (kp) | 11-13 |
| Disaggregation time (min) | 11' |

### EXAMPLE 2

### Preparation of Indapamide Controlled Release Layer

### Composition of the Layer

| | | |
|---|---|---|
| Indapamide | 1.5 | mg |
| Lactose monohydrate | 124.5 | mg |
| Hydroxypropylmethylcellulose | 63.8 | mg |
| Povidone K30 | 8.6 | mg |
| Colloidal silicon dioxide | 0.4 | mg |
| Magnesium stearate | 1.2 | mg |

### Procedure

Indapamide, lactose monohydrate, hypromellose, povidone and colloidal silicon dioxide were sieved and blended (through geometric dilution) for few minutes.

Magnesium stearate was sieved, added and blended for few minutes with the above mixture.

After blending, final mixture was compressed to the target weight (200 mg) as the first layer of the double layer tablet, to the hardness range of 3-6 kp.

### Technological Controls

Technological controls of the mixture and of the tablets are reported in the table below:

Technological characterization: granule

| | | |
|---|---|---|
| Compressibility Index (%) | 18.03 | (fair) |
| Hausner Ratio | 1.23 | (fair) |

Technological characterization: tablet

| | |
|---|---|
| Hardness (Kp) | 3-5 |

### EXAMPLE 3

### Manufacturing Process

The manufacturing process of the dual-layer tablet is reported in the flow chart.

As can be seen below first is prepared the layer of indapamide modified release and then is compressed, as second layer, the immediate release layer of losartan potassium.

The characteristics of the tablets obtained are reported in the table:

| | Losartan potassium/Indapamide strengh | |
|---|---|---|
| | 100/1.5 mg | 50/1.5 mg |
| Components 1° Layer | Quantity mg/tbs) | |
| Indapamide final mixture | 200 | 200 |
| Components 2° Layer | Quantity (mg/tbs) | |
| Losartan potassium final mixture | 300 | 150 |
| Theoretical tablet weight (mg) | 500 | 350 |
| Tool | Round biconvex, | Round biconvex, |
| | 10.0 mm | 9.0 mm |

| | |
|---|---|
| Manufacturing method | Dual-layer tableting machine |

Technological characterization: 1° layer

| | | |
|---|---|---|
| Hardness (kp) | 3-6 | 3-6 |
| Thickness (mm) | 3.20 | 3.60 |

Technological characterization: double layer tablet

| | | |
|---|---|---|
| Hardness (kp) | 18-22 | 12-15 |
| Thickness (mm) | 5.95 | 5.20 |

### EXAMPLE 4

### In Vitro Release

The dissolution was tested using the method of USP apparatus (paddle) 50 rpm; Medium: buffer solution pH 6.8; Volume 900 ml; Sampling time.

10, 20, 30, 45, 60 (min) withdrawal for losartan potassium analysis

2, 6, 12, 24 (hours) withdrawal for indapamide analysis

Dissolution test performed on dual layer tablets of example 3 vs. the single commercial references Lortaan® (for losartan potassium) and Natrilix® LP (for indapamide modified release) is reported in the table below:

| | | Losartan | Samp- | Natrilix | |
|---|---|---|---|---|---|
| Sampling | Lortaan | potassium | ling | LP 1.5 mg | Indap- |
| times | 100 mg tbs | 100 mg | times | tbs, (Ref- | amide |
| (minutes) | (Reference) | Example 3 | (hours) | erence) | Example 3 |
| % Losartan potassium release | | | % Indapamide release | | |
| 0 | 0 | 0 | 0 | 10 | 0 |
| 10 | 32 | 32 | 2 | 7 | 10 |
| 20 | 62 | 60 | 6 | 20 | 23 |
| 30 | 83 | 83 | 12 | 37 | 40 |
| 45 | 97 | 101 | 24 | 64 | 60 |
| 60 | 98 | 103 | | | |

As it can be seen form the comparison and the similarity factor F2 (75.7 for losartan, and 72.8 for indapamide) the release profiles of the single references are comparable with those obtained with dual layer tablets.

### EXAMPLE 5

### Stability

The stability of the formulations prepared as described in Examples 1 to 3 was checked for assay, related substances and dissolution after 3 months at room temperature and in accelerated conditions, according to international Conference on Harmonisation (ICH), and there were no significant changes from initial results.

### EXAMPLE 6

### Capsule Formulation

The association of potassium losartan and indapamide can be carried out with a capsule formulation containing the two active ingredients. Potassium losartan is formulated as an immediate release particle and indapamide as a prolonged release minitablets that can fit into a capsule.

A) Preparation of Losartan Granulate

Losartan potassium (33%), cellulose microcrystalline (35% wt), lactose monohydrate (17% wt) and part of pregelatinized maize starch (7% wt) were sieved and blended for few minutes.

The mixture of powders was kneaded using a solution of purified water and pregelatinized maize starch (remaining 7% wt), and the wet granulate was passed through a 2.0 mm sieve.

The granulate was dried in a static oven at 50° C. until a LOD≦4% is reached, then passed through a 0.6 mm sieve.

Silicon dioxide (0.1% wt) was sieved and added to facilitate flowability.

B) Preparation of Indapamide Controlled Release Mini Tablets

Indapamide (0/5% wt), lactose monohydrate (62.2% wt), hypromellose (32% wt), povidone (4.3% wt) and colloidal silicon dioxide (0.2% wt) and magnesium stearate (0.6% wt) were sieved and blended (through geometric dilution) for few minutes.

The mixture was compressed with round biconvex punches of 5.0 mm diameter to the target weight (100 mg), with hardness of 10 kp.

C) Preparation of the Capsules

Capsules of size zero are filled with 2 round minitablets of indapamide and the granulate of potassium losartan (300 mg).

### EXAMPLE 7

### Dry Coated Tablets

The association of potassium losartan and indapamide can be performed with a dry coating technology. The prolonged release minitablets of indapamide are dry coated with the fast release granulate of potassium losartan; namely leading to a minitablet inside a bigger tablet.

A) Preparation of the Indapamide Minitablet

| | | |
|---|---|---|
| Indapamide | 0.75 | mg |
| Lactose monohydrate | 62.25 | mg |
| Hydroxypropylmethylcellulose | 31.9 | mg |
| Povidone K30 | 4.3 | mg |
| Colloidal silicon dioxide | 0.2 | mg |
| Magnesium stearate | 0.6 | mg |

### Procedure

Indapamide, lactose monohydrate, hypromeliose, povidone and colloidal silicon dioxide were sieved and blended (through geometric dilution) for few minutes.

Magnesium stearate was sieved, added and blended for few minutes with the above mixture.

After blending, final mixture was compressed to the target weight (100 mg)

B) Preparation of the Losartan Granulate

| | | |
|---|---|---|
| Losartan potassium micronized | 100.0 | mg |
| Lactose monohydrate | 65 | mg |
| Cellulose microcrystalline | 128.0 | mg |
| PVP VA 65 | 15.0 | mg |
| Magnesium stearate | 2.0 | mg |

### Procedure

Losartan potassium, cellulose microcrystalline, lactose monohydrate and magnesium stearate were sieved and blended for few minutes and dry compacted in 1 g slugs. The slugs were milled and the obtained granulate was used for dry coating the indapamide minitablets previously described.

C) Dry Coated Tablets

The minitablets and the losartan granulate were loaded into a Kilian /IMA SD 250 tablet press machine to obtain dry coated tablets.

1. The combination of fast release ANGIOTENSIN II AT-1 RECEPTOR ANTAGONIST with a controlled release diuretic.

2. The combination, according to claim 1 wherein the ANGIOTENSIN II AT-1 RECEPTOR ANTAGONISTS inhibitor is losartan potassium and the diuretic is indapamide.

3. A dosage form comprising an ANGIOTENSIN II AT-1 RECEPTOR ANTAGONISTS inhibitor and a diuretic.

4. A dosage form according to claim 3 for achieving a fast release of the ANGIOTENSIN II AT-1 RECEPTOR ANTAGONISTS inhibitor and a controlled release of the diuretic.

5. A dosage form according to claim 3 in which the ANGIOTENSIN II AT-1 RECEPTOR ANTAGONISTS inhibitor is losartan potassium and the diuretic is indapamide.

6. A dosage form according to any claim 3 coated with one or more polymers or pharmaceutically acceptable seal coat polymers.

7. A dosage form according to claim 3 selected from a capsule for oral administration and a dual layer tablet.

8. The combination of an ANGIOTENSIN II AT-1 RECEPTOR ANTAGONISTS inhibitor with a controlled release diuretic for use in the treatment of cardiovascular and blood pressure related disorders.

9. The combination according to claim 8 in which the ANGIOTENSIN II AT-1 RECEPTOR ANTAGONIST is in a fast release form.

10. The combination of claim 1 wherein the fast release compound comprises 10 to 60 wt % diluent, 0.01 to 5 wt % lubricants, 1 to 20 wt % binders, and 0.1 to 20 wt % disintegrants.

11. The combination according to claim 1 in which the diluent is selected from lactose, mannitol, starch and microcrystalline cellulose.

12. The combination according to claim 1 in which the lubricant is selected from magnesium stearate, talc and colloidal silicon dioxide.

13. The combination according to claim 1 in which the binder is selected from providone, modified starch and polyvinyl alcohol.

14. The combination according to claim 1 in which the distintegrant is sodium carboxymethylcellulose.

15. The combination according to claim 8 wherein indapamide and losartan potassium are present in a total amount between 0.1 and 40% w/w of the formulation.

16. The combination according to claim 1 wherein the controlled diuretic comprises a controlled release layer which is controlled with cellulose derivatives.

17. The combination according to claim 16 wherein the cellulose derivatives is hydroxypropylmethyl cellulose.

18. The combination according to claim 1 for use in oral administration at a dose in the range of 10 to 200 mg/day of losartan and 0.5 to 5 mg/day of indapamide.

19. A combination according to claim 1 and a pharmaceutically acceptable carrier/diluent.

20. A process for the preparation of a combination according claim 1 comprising:
(i) preparing a first layer containing a diuretic mixing with pharmaceutically acceptable additives,
(ii) tableting the mixture of step (i)
(iii) preparing a second layer containing an ANGIOTENSIN II AT-1 RECEPTOR ANTAGONISTS inhibitor mixing with pharmaceutically acceptable additives and granulating the mixture
(iv) compressing in a single dual-layer tablet the granulated and dried mixture of step (iii) with the tablets of step (ii).

21-25. (canceled)
